(19) United States
(12) Patent Application Publication
(10) Pub. No.: US 2026/0002844 A1
(43) Pub. Date: Jan. 1, 2026

(54) DEVICE FOR ON-SITE CUTTING AND TRANSFERRING OF NATURAL GAS HYDRATE SAMPLE

(71) Applicant: GUANGZHOU INSTITUTE OF ENERGY CONVERSION, CHINESE ACADEMY OF SCIENCES, Guangzhou (CN)

(72) Inventors: Xiaosen LI, Guangzhou (CN); Yi WANG, Guangzhou (CN); Jianwu LIU, Guangzhou (CN); Rui XU, Guangzhou (CN); Zhaoyang CHEN, Guangzhou (CN); Gang LI, Guangzhou (CN); Yu ZHANG, Guangzhou (CN)

(73) Assignee: GUANGZHOU INSTITUTE OF ENERGY CONVERSION, CHINESE ACADEMY OF SCIENCES, Guangzhou (CN)

(21) Appl. No.: 19/011,672

(22) Filed: Jan. 7, 2025

Related U.S. Application Data
(63) Continuation of application No. PCT/CN2024/103908, filed on Jul. 5, 2024.

(30) Foreign Application Priority Data
Jun. 28, 2024 (CN) 202410861144.X

(51) Int. Cl. G01N 1/08; G01N 33/24

(52) U.S. Cl. CPC G01N 1/08; G01N 33/24

(57) ABSTRACT

A device for on-site cutting and transferring of natural gas hydrate sample includes a servo transmission mechanism, a pressure-resistant manipulator, a high-pressure and sealing operating mechanism, a pressure-maintaining unit, and a data acquisition system; when working, the servo transmission mechanism is used for driving the pressure-resistant manipulator to reciprocating motion; a servo rotating mechanism is used for realizing 360-degree rotation of a tightly clamped sample; the pressure-resistant manipulator is used for driving a grabber to grab the sample, and cooperates with the high-pressure and sealing operating mechanism to assist a cutting unit in cutting the sample, and the reciprocating motion is achieved by the servo transmission mechanism; the cut sample is placed in a high-pressure and pressure-maintaining bin for storage; a system pressure is acquired by a pressure acquisition system, and acquired data is transmitted to a data acquisition and control system for data processing and analyzing.

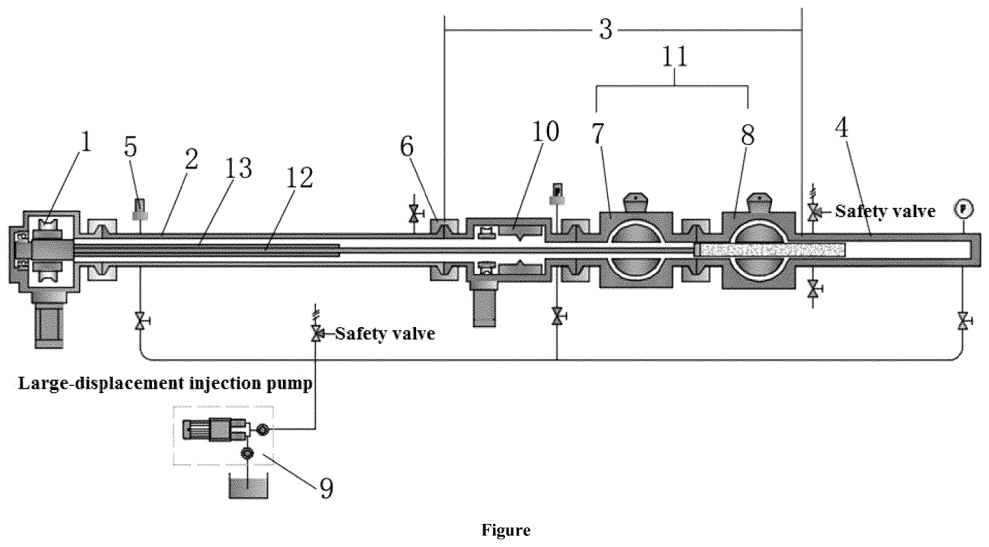

### CROSS REFERENCE TO THE RELATED APPLICATIONS

This application is a continuation application of International Application No. PCT/CN2024/103908, filed on Jul. 5, 2024, which is based upon and claims priority to Chinese Patent Application No. 202410861144.X, filed on Jun. 28, 2024, the entire contents of which are incorporated herein by reference.

### TECHNICAL FIELD

The present invention relates to the technical field of natural gas hydrate exploitation, in particular to a device for on-site cutting and transferring of natural gas hydrate sample.

### BACKGROUND

It is considered that the deep-sea oil and gas resource realm is an important frontier territory of the petroleum industry, and deep-water and ultra-deep-water oil and gas resources have become a hot spot for the United States, Britain and other western developed countries to compete for exploitation. A main natural gas hydrate resource buried in the sea at a depth of 1-3 km is generally regarded as an unconventional oil and gas resource with huge untapped reserves, and is one of the most potential alternative energy sources in the 21st century. Its huge resource potential has attracted countries all over the world to deepen their work in exploration, experimental exploitation, and supporting environmental impact assessment. The United States, Canada, Germany and Norway, as well as countries around China such as Japan, India, South Korea and Vietnam have all formulated long-term research plans for natural gas hydrates.

With the in-depth study of the research on the natural gas hydrate, a number of experimental devices have been developed to simulate a generation and exploitation process of the natural gas hydrate. However, with the continuous deepening of the research, requirements for such experimental devices are getting higher and higher. At present, the requirements for the experimental simulation devices of the natural gas hydrate at home and abroad are approximately as follows.
(1) Experiments are becoming more and more comprehensive.

With the continuous deepening of scientific research, scientific research has become unitary, interdisciplinary and integrated. The design requirements for the experimental simulation devices of the natural gas hydrate have also evolved from original single functional requirements to the integrated and systematic design requirements. According to the investigation, it is found that the current experimental simulation devices of the natural gas hydrate can simulate not only the phase state relationship of the hydrate but also the sedimentary strata of the hydrate, test various physical and chemical properties of the stratum when the hydrate is generated or not, and study the stratum parameter characteristics and the like of the hydrate.
(2) The degree of visualization is getting higher and higher.

After the development of macro science to a certain extent, it is not enough to explain the active physical phenomena. In order to analyze and understand various mechanism characteristics of the natural gas hydrate as clearly as possible, higher functional requirements are put forward for an experimental simulation system, hoping to acquire the phase state change processes of the generation, aggregation and decomposition of the natural gas hydrate in real time through the visualization technology. It is even possible to observe and record the micro-world of an object to be researched through other testing equipment.
(3) Environmental conditions simulated by the experiments are becoming stricter and stricter.

With the deepening of sampling of the natural gas hydrate on the spot, it is becoming more and more clear about the environment in which the natural gas hydrate is accumulated, and new challenges are posed to the experimental simulation devices. Huge stratum pressure and lower ambient temperature are necessary conditions for the generation of the natural gas hydrate. In order to create these conditions, higher requirements have been put forward for an experimental simulation system. How to accurately control the simulation environment, how to ensure the normal operation of each experimental device under harsh environmental conditions, and how to ensure the higher structural reliability of the experimental devices under such environmental conditions are focuses of the design and research of the experimental system.
(4) The measurement accuracy is getting higher and higher.

In order to accurately explain the accumulation state of the natural gas hydrate and the influence of exploitation methods on the stratum, truly reflect the various characteristics of the natural gas hydrate in the stratum, and understand and master various mechanism relationships, higher requirements are put forward for the testing ability of the experimental simulation system. High-precision measurement and high-precision control are the necessary prerequisites for accurate simulation. For achieving this purpose, in addition to the selection of reliable and accurate sensors, higher requirements are also put forward for the design solution. How to reasonably arrange and use various components and equipment such as sensors is also a focus that affects the measurement accuracy of the experimental simulation device.

Undersea gas hydrate reservoir is a multi-phase and multi-component complex sediment system composed of natural gas, water, hydrate, ice, sand, etc., and different gas hydrate reservoir conditions will cause great differences in exploitation technology methods and exploitation results. Therefore, the on-site characterization technology of key geological parameters of a gas hydrate target reservoir is one of the most critical technical means to detect gas hydrate before exploitation, and it is also the basis for further accurately predicting a gas hydrate production capacity in a target region and determining an exploitation solution. At present, the on-site parameter characterization and the online production capacity evaluation of the marine gas hydrate reservoir have become key technical issues in commercial gas hydrate exploitation, and it is urgent to carry out special research and development and establish a mature technology and special device system.

However, in the prior art, there are following technical difficulties: at present, there is no device in the prior art yet that can realize any-size cutting, transferring and segmented storage of a gas hydrate undersea in-situ pressure-maintaining sample under a pressure maintenance condition.

### SUMMARY

The present invention aims at solving the technical difficulties existing in the prior art, and provides a device for on-site cutting and transferring of natural gas hydrate sample.

In order to solve the problems existing in the prior art, the present invention adopts the following technical solutions.

A device for on-site cutting and transferring of natural gas hydrate sample includes a servo transmission mechanism, a pressure-resistant manipulator, a high-pressure and sealing operating mechanism, a pressure-maintaining unit and a data acquisition system;

wherein the servo transmission mechanism is sequentially connected to the pressure-resistant manipulator, the high-pressure and scaling operating mechanism and the pressure-maintaining unit, wherein the high-pressure and sealing operating mechanism includes a cutting unit and a sealing unit; and

when the device for on-site cutting and transferring of natural gas hydrate sample is working, the servo transmission mechanism drives a sample to reciprocating motion or rotate in the pressure-resistant manipulator, after the sample enters a high-pressure and sealing operating unit, the sample is cut by the cutting unit, and the cut sample enters the pressure-maintaining unit to be stored.

As an improvement of the technical solution of the device for on-site cutting and transferring of the natural gas hydrate sample, the servo transmission mechanism includes a servo motor, a servo driver, a servo rotating mechanism and a sample extractor which are sequentially connected; and

the sample extractor is used for gripping the sample, and the servo motor is used for driving the servo driver to drive the sample extractor to reciprocating motion in the pressure-resistant manipulator, and driving the servo rotating mechanism to drive the sample extractor to rotate in the pressure-resistant manipulator.

As an improvement of the technical solution of the device for on-site cutting and transferring of the natural gas hydrate sample, the servo transmission mechanism further includes a servo controller, and the servo controller is connected to both the servo driver and the servo rotating mechanism.

As an improvement of the technical solution of the device for on-site cutting and transferring of the natural gas hydrate sample, the pressure-resistant manipulator is capable of withstanding pressures from 0 to 40 MPa; and

the whole pressure-resistant manipulator is sleeve-shaped, a sheath is arranged outside the sample, and the servo transmission mechanism drives the sample and the sheath to reciprocating motion and rotate in the pressure-resistant manipulator.

As an improvement of the technical solution of the device for on-site cutting and transferring of the natural gas hydrate sample, the high-pressure and sealing operating unit is detachable connected to the pressure-resistant manipulator, the cutting unit is detachable connected to the sealing unit, and the sealing unit is detachable connected to the pressure-maintaining unit.

As an improvement of the technical solution of the device for on-site cutting and transferring of the natural gas hydrate sample, the sealing unit includes a plurality of ball valves; and

the plurality of ball valves includes a first ball valve and a second ball valve, the first ball valve is arranged between the cutting unit and the pressure-resistant manipulator, and the second ball valve is arranged between the cutting unit and the pressure-maintaining unit.

As an improvement of the technical solution of the device for on-site cutting and transferring of the natural gas hydrate sample, the device for on-site cutting and transferring of the natural gas hydrate sample further includes a pressurizing system for maintaining and stabilizing an internal pressure of the device for on-site cutting and transferring of the natural gas hydrate sample.

As an improvement of the technical solution of the device for on-site cutting and transferring of the natural gas hydrate sample, the data acquisition system includes a pressure acquisition system and a temperature acquisition system, the pressure acquisition system includes pressure sensors and a pressure acquisition module, and the temperature acquisition system includes temperature sensors and a temperature acquisition module; and

the plurality of pressure sensors and the plurality of temperature sensors acquire real-time pressure values and temperature values of different positions in the device for on-site cutting and transferring of the natural gas hydrate sample.

As an improvement of the technical solution of the device for on-site cutting and transferring of the natural gas hydrate sample, the data acquisition system further includes a computer, and data processing and analyzing software installed in the computer.

The present invention has the following beneficial effects.
1. According to the present invention, the key technical difficulties of in-situ processing and analysis of a core drilled in the gas hydrate reservoir on the site are broken through; the analysis and the research of the natural gas hydrate at the site scale are achieved; the portability of gas hydrate analysis equipment is greatly improved; measurement modes (referring to key technical parameters in exploitation of gas hydrate such as a gas hydrate decomposition rate, a gas-liquid flow rate, a sediment shape variable and decomposed gas compositions) are upgraded; and a 1 m gas hydrate core sample under 30 MPa can be directly processed.
2. In the present invention, the laboratory testing technology is extended to on-site testing, which will directly provide important theoretical basis and key design parameters for the research and development of on-site reservoir parameter characterization and on-line production capacity prediction devices that must be established for the industrialization of the gas hydrate in the future, fill the gap that there is no special engineering equipment for on-site testing of the gas hydrate in China, and provide important theoretical support and technical support for the industrialization process of the gas hydrate in China.
3. In the present invention, under the joint action of the servo transmission mechanism, a pressure-stabilizing manipulator, the high-pressure and scaling operating mechanism and the pressure-maintaining unit which are sequentially arranged, effects of transferring, cutting and segmented storage of the marine gas hydrate sample under the high-pressure and pressure-maintaining state are achieved, and the problem that there is no device in the prior art yet that can realize any-size cutting, transferring and segmented storage of the gas hydrate seabed in-situ pressure-maintaining sample under the pressure maintenance condition is solved.

### BRIEF DESCRIPTION OF THE DRAWINGS

Figure is a schematic structural diagram of the present invention.

Description of reference numerals: 1, servo transmission mechanism; 2, pressure-resistant manipulator; 3, high-pressure and sealing operating mechanism; 4, pressure-maintaining unit; 5. data acquisition system; 6, clamp; 7, first ball valve; 8, second ball valve; 9, pressurizing system; 10, cutting unit; 11, sealing unit; 12, sample; and 13, sheath.

### DETAILED DESCRIPTION OF THE EMBODIMENTS

To make the objectives, technical solutions and beneficial effects of the present invention clearer, the technical solutions in the embodiments of the present invention will be clearly and completely described below with reference to the accompanying drawings in the embodiments of the present invention. Obviously, the embodiments described are merely some but not all embodiments of the present invention.

As shown in the figure, a device for on-site cutting and transferring of a natural gas hydrate sample includes a servo transmission mechanism 1, a pressure-resistant manipulator 2, a high-pressure and sealing operating mechanism 3, a pressure-maintaining unit 4 and a data acquisition system 5.

The servo transmission mechanism 1 is sequentially connected to the pressure-resistant manipulator 2, the high-pressure and scaling operating mechanism 3 and the pressure-maintaining unit 4.

The high-pressure and sealing operating mechanism 3 includes a cutting unit 10 and a sealing unit 11.

When the device for on-site cutting and transferring of natural gas hydrate sample is working, the servo transmission mechanism 1 drives a sample 12 to reciprocating motion or rotate in the pressure-resistant manipulator 2, after the sample 12 enters a high-pressure and scaling operating unit, the sample 12 is cut by the cutting unit 10, and the cut sample 12 enters the pressure-maintaining unit 4 to be stored.

Specifically, the device according to the present invention is mainly used to realize effects of any-size cutting, transferring and segmented storage of a gas hydrate seabed in-situ pressure-maintaining sample 12 under a pressure maintenance condition. In the present invention, under the joint action of the servo transmission mechanism 1, a pressure-stabilizing manipulator, the high-pressure and sealing operating mechanism 3 and the pressure-maintaining unit 4 which are sequentially arranged, the effects of transferring, cutting and segmented storage of the marine gas hydrate sample 12 under the high-pressure and pressure-maintaining condition are achieved.

In the present invention, the accumulation of the natural gas hydrate in nature can be directly studied, and in-situ processing and analysis of a core drilled in the gas hydrate reservoir on the site is achieved; and the device can be mounted on a gas hydrate exploration vessel or a drilling vessel, and is extremely high in portability. Meanwhile, according to the present invention, the miniaturized design meets the quickness and portability of the cutting and segmented storage of the gas hydrate under the high-pressure and pressure-maintaining condition.

More specifically, in the present invention, the servo transmission mechanism 1 is used for driving the sample 12 to reciprocating motion or rotate in the pressure-resistant manipulator 2, including pushing the sample 12 to move in the pressure-resistant manipulator 2, so as to adjust the length and/or angle of the sample 12 in the present invention. The pressure-resistant manipulator 2 is used for separating the sample 12 under a pressure, that is, a separation effect of the sample 12 and the sheath 13 is completed under the high-pressure and pressure-maintaining state through the pressure-resistant manipulator 2.

The high-pressure and sealing operating mechanism 3 includes the cutting unit 10 and the sealing unit 11. In the present invention, the cutting unit 10 achieves the cutting and segmentation effect on the sample 12, and the sealing unit 11 maintains the overall pressure of the device in the present invention, so that an adverse influence of pressure leakage on the quality of the marine gas hydrate sample 12 is avoided. Under the action of the servo transmission mechanism 1, the sample 12 cut in the high-pressure and pressure-maintaining state is transferred to the pressure-maintaining unit 4, allowing the cut sample 12 to be stored.

It should be noted herein that the formation of the gas hydrate requires four basic conditions: low temperature, high pressure environment, a sufficient gas source and sufficient water. In the present invention, it is necessary to ensure the high pressure, pressure maintaining and sealing state of the device for on-site cutting and transferring of the natural gas hydrate sample.

Meanwhile, in the present invention, data such as pressures and temperatures can be further detected and acquired in real time through the data acquisition system 5, so as to ensure the high pressure, pressure maintenance and sealing state of the device for on-site cutting and transferring of the natural gas hydrate sample in the present invention and ensure the storage environment of the gas hydrate sample 12.

In some embodiments of the present invention, the cutting unit 10 is used for performing online isobaric cutting on the sample 12, and cutting the sheath 13 of the sample 12 and the sample 12; and rotational displacement can be read, so that the in-situ cutting can be achieved without damaging the reservoir sample 12.

Specifically, the cutting unit 10 is mainly used for cutting the sample 12 into segments. Since the sample 12 when sampling is stored in a PC tube, the external PC tube of the sample 12 needs to be cut first, and then the sample 12 is cut. The sample 12 is designed with two sets of cutting tools, which are controlled by one set of manual feeding mechanism and which can switch different blades by clockwise and anticlockwise rotation, and the rotational displacement can be read through a counter. As a specific example of this embodiment, the cutting unit 10 can withstand pressures from 0 to 40 MPa, has the working temperatures from −10° C. to 60° C., and adopts 316 L stainless steel as a wetting medium.

In some embodiments of the present invention, the servo transmission mechanism 1 includes a servo motor, a servo driver, a servo rotating mechanism and a sample extractor 12 which are sequentially connected.

The sample extractor 12 is used for gripping the sample 12, and the servo motor is used for driving the servo driver to drive the sample extractor 12 to reciprocating motion in the pressure-resistant manipulator 2, and driving the servo rotating mechanism to drive the sample extractor 12 to rotate in the pressure-resistant manipulator 2.

Preferably, the servo transmission mechanism 1 is further designed with a decelerating mechanism which can achieve self-locking when power-off; and the driver is designed with a pressure balance mechanism to achieve the isobaric work of a driving shaft.

Preferably, the servo transmission mechanism 1 is further equipped with a servo controller. The servo controller is connected to both the servo driver and the servo rotating mechanism, and may be further connected to a computer to achieve the effect of online control. As a specific example of this embodiment, the servo motor is imported, with the power of 1 KW, rated speed of 3, 000 r/min, maximum speed of 5, 000 r/min, rated torque of 2.39 N·m, and maximum torque of 7.16 N·m.

In some embodiments of the present invention, the pressure-resistant manipulator 2 can withstand pressures from 0 to 40 MPa. The whole pressure-resistant manipulator 2 is sleeve-shaped, the sheath 13 is arranged outside the sample 12, and the servo transmission mechanism 1 drives the sample 12 and the sheath 13 to reciprocating motion and rotate in the pressure-resistant manipulator 2.

Specifically, based on the requirements of the storage environment of the gas hydrate, it is necessary to keep the gas hydrate in a high-pressure and pressure-maintaining environment, and thus, all components in the present invention should have the high-pressure resistance.

In the present invention, the pressure-resistant manipulator 2 can withstand pressures from 0 to 40 MPa. The pressure-resistant manipulator 2 includes a high-pressure and pressure-maintaining bin connected to the servo transmission mechanism 1. The high-pressure and pressure-maintaining bin is sleeve-shaped, so that the sample 12 and the sheath 13 can conveniently reciprocating motion or rotate in the pressure-resistant manipulator 2. The end of the high-pressure and pressure-maintaining bin is connected to a separator of a core holder, and is sealed by the separator of the core holder.

As a specific example of the present invention, the pressure-resistant manipulator 2 can withstand the pressure of 35 MPa, is made of 316 L stainless steel, and can achieve on-line displacement control with the control accuracy of ±0.5 cm.

Further, the high-pressure and sealing operating mechanism 3 further includes a sealing cavity, in which a spring clamping mechanism is arranged. The sample 12 transferred from the pressure-resistant manipulator 2 is clamped and fixed by the spring clamping mechanism, such that the cutting of the cutting unit 10 is facilitated and the cutting accuracy is ensured.

Furthermore, the high-pressure and sealing operating mechanism 3 further includes a manual rotating mechanism and a spring clamping mechanism, which are both designed and mounted in the sealing cavity; and the overall sealing cavity can guarantee the pressure resistance of 30 MPa. The manual rotating mechanism is of a combined structure of a worm gear and a lead screw nut mechanism, and a gear and a high-pressure sealing cavity body realize high-pressure rotating sealing; and the manual rotating mechanism is driven by an external handle. Meanwhile, a lead screw is designed on a worm, and a lead screw nut and a spring reset mechanism are designed on the spring clamping mechanism.

In some embodiments of the present invention, the pressure-maintaining unit 4 is used for performing pressure tracking on the pressure-resistant manipulator 2, the sealing cavity and a pressure-maintaining storage bin. As a specific example of the present invention, the pressure-maintaining unit 4 adopts multiple groups of superchargers that work cooperatively, the control pressure is 30 MPa, and the control accuracy of the transfer pressure of the sample 12 is 5%.

In some embodiments of the present invention, the high-pressure and sealing operating unit is detachable connected to the pressure-resistant manipulator 2, the cutting unit 10 is detachable connected to the sealing unit 11, and the sealing unit 11 is detachable connected to the pressure-maintaining unit 4.

In the present invention, the high-pressure and sealing operating unit and the pressure-resistant manipulator 2, the cutting unit 10 and the sealing unit 11, and the sealing unit 11 and the pressure-maintaining unit 4 are all connected by detachable connecting parts which may be flanges or clamps 6. In order to facilitate the detachable connection between the components, preferably, the connecting part is the clamp 6, which can improve the convenience of the connection.

In some embodiments of the present invention, the sealing unit 11 includes a plurality of ball valves. The plurality of ball valves includes a first ball valve 7 and a second ball valve 8. The first ball valve 7 is arranged between the cutting unit 10 and the pressure-resistant manipulator 2, and the second ball valve 8 is arranged between the cutting unit 10 and the pressure-maintaining unit 4.

As a specific example of the present invention, in the present invention, the pressure-resistant manipulator 2 is connected to the cutting unit 10 in the high-pressure and sealing operating mechanism 3 through the clamp 6, and the cutting unit 10 is connected to the first ball valve 7 through the clamp 6. The second ball valve 8 is arranged between the cutting unit 10 and the pressure-maintaining unit 4, and the first ball valve 7 and the second ball valve 8 are connected through the clamp 6.

In some embodiments of the present invention, the device for on-site cutting and transferring of the natural gas hydrate sample further includes a pressurizing system 9 for maintaining and stabilizing an internal pressure of the device for on-site cutting and transferring of the natural gas hydrate sample.

In some embodiments of the present invention, the data acquisition system 5 includes a pressure acquisition system and a temperature acquisition system. The pressure acquisition system includes pressure sensors and a pressure acquisition module, and the temperature acquisition system includes temperature sensors and a temperature acquisition module.

The plurality of pressure sensors and the plurality of temperature sensors acquire real-time pressure values and temperature values of different positions in the device for on-site cutting and transferring of the natural gas hydrate sample. Further, the data acquisition system 5 further includes a computer, and data processing and analyzing software installed in the computer.

Specifically, the data acquisition system 5 includes a pressure acquisition system and a temperature acquisition system. The pressure acquisition system includes the pressure sensors and the pressure acquisition module, and the temperature acquisition system includes the temperature sensors and the temperature acquisition module. The temperature sensor is used for measuring a temperature of each point in the present invention and transmitting the measured temperature information to the temperature acquisition module. The pressure sensor is used for measuring a pressure of each point in the present invention and transmitting the measured pressure information to the pressure acquisition module, so that the temperature information can be subsequently fed back to the data analyzing and processing software of the computer by the temperature acquisition module and is analyzed and processed.

As a specific example of the present invention, the temperature sensor module adopts a high-precision temperature sensor, with the temperature measuring range from −50° C. to 60° C. and the accuracy of 0.5%. The pressure sensor adopts a high-precision differential pressure sensor from Senex Company, with the measuring range of 30 MPa and the accuracy of 0.1% FS; and there are 7 sets of high-precision differential pressure sensors. The acquisition module is mainly used for acquiring pressure sensor data in a system flow, and the acquired data is fed back to the software through the acquisition module.

Further, the data acquisition system 5 further includes a computer, and data processing and analyzing software installed in the computer.

In some embodiments of the present invention, the data acquisition system 5, as in the prior art, includes the computer, and the data processing and analyzing software installed in the computer. A data acquisition and processing system is mainly composed of acquisition hardware and acquisition software. The acquisition hardware is mainly composed of a data acquisition card. a PLC controller and the computer. The acquisition software is based on Win7 system simulation software and system analyzing and testing software. Therefore, all-round detection of the pressure and the flow are achieved; the precise control and the interface control are achieved; and data analysis and real-time data analysis of various seepage treatment reports, curves and the like are achieved.

As a specific example of the present invention, the device for on-site cutting and transferring of the natural gas hydrate sample includes the servo transmission mechanism 1, a pressure-stabilizing manipulator, the high-pressure and sealing operating mechanism 3 and the pressure-maintaining unit 4 which are sequentially arranged. The high-pressure and sealing operating mechanism 3 includes the cutting unit 10 and the sealing unit 11. Under the joint action of the servo transmission mechanism 1, the pressure-stabilizing manipulator, the high-pressure and sealing operating mechanism 3 and the pressure-maintaining unit 4 which are sequentially arranged, the effects of transferring, cutting and segmented storage of the marine gas hydrate sample 12 under the high-pressure and pressure-maintaining condition are achieved.

When device for on-site cutting and transferring of the natural gas hydrate sample is working, the servo transmission mechanism 1 is used for driving the pressure-resistant manipulator 2 to reciprocating motion, so as to realize the 360-degree rotation of the sample 12 after the sample 12 is tightly clamped. The pressure-resistant manipulator 2 is used for driving a grabber to grab the sample 12, and cooperates with the high-pressure and sealing operating mechanism 3 to assist the cutting unit 10 for the sample 12 in cutting the sample 12; and the reciprocating motion is achieved under the action of the servo transmission mechanism 1. The cut sample 12 is placed in the high-pressure and pressure-maintaining bin in the pressure-maintaining unit 4 for storage. During the whole cutting process, a system pressure is acquired by a pressure acquisition system, and acquired data is transmitted to a data acquisition and control system for data processing and analyzing.

Since the high-pressure and sealing operating unit is detachable connected to the pressure-resistant manipulator 2, the cutting unit 10 is detachable connected to the sealing unit 11, and the sealing unit 11 is detachable connected to the pressure-maintaining unit 4, by opening or closing the first ball valve 7 and the second ball valve 8, the on-site assembling of the device according to present invention can be achieved, and a transferring effect of the sample 12 in the high-pressure and pressure-maintaining state can be ensured conveniently.

All other embodiments obtained by those of ordinary skills in the art without creative efforts based on the embodiments in the present invention are within the protection scope of the present invention.

What is claimed is:

1. A device for on-site cutting and transferring of a natural gas hydrate sample, comprising:
a servo transmission mechanism, a pressure-resistant manipulator, a high-pressure and sealing operating mechanism, a pressure-maintaining unit, and a data acquisition system, wherein
the servo transmission mechanism is sequentially connected to the pressure-resistant manipulator, the high-pressure and sealing operating mechanism, and the pressure-maintaining unit, wherein the high-pressure and sealing operating mechanism comprises a cutting unit and a sealing unit; and
when working, the servo transmission mechanism drives the natural gas hydrate sample to perform reciprocating motion or rotate in the pressure-resistant manipulator, and when the natural gas hydrate sample enters a high-pressure and sealing operating unit, the natural gas hydrate sample is cut by the cutting unit, and a cut sample enters the pressure-maintaining unit to be stored.

2. The device according to claim 1, wherein the servo transmission mechanism comprises a servo motor, a servo driver, a servo rotating mechanism, and a sample extractor, wherein the servo motor, the servo driver, the servo rotating mechanism, and the sample extractor are sequentially connected; and
the sample extractor is used for gripping the natural gas hydrate sample, and the servo motor is used for driving the servo driver to drive the sample extractor to perform reciprocating motion in the pressure-resistant manipulator, and driving the servo rotating mechanism to drive the sample extractor to rotate in the pressure-resistant manipulator.

3. The device according to claim 2, wherein the servo transmission mechanism further comprises a servo controller, and the servo controller is respectively connected to the servo driver and the servo rotating mechanism.

4. The device according to claim 1, wherein the pressure-resistant manipulator is configured for withstanding pressures from 0 to 40 MPa; and
the pressure-resistant manipulator is sleeve-shaped, a sheath is arranged outside the natural gas hydrate sample, and the servo transmission mechanism drives the natural gas hydrate sample and the sheath to perform reciprocating motion and rotate in the pressure-resistant manipulator.

5. The device according to claim 1, wherein the high-pressure and sealing operating unit is detachably connected to the pressure-resistant manipulator, the cutting unit is detachably connected to the sealing unit, and the sealing unit is detachably connected to the pressure-maintaining unit.

6. The device according to claim 5, wherein the sealing mechanism comprises a plurality of ball valves; and
the plurality of ball valves comprise a first ball valve and a second ball valve, wherein the first ball valve is arranged between the cutting unit and the pressure-resistant manipulator, and the second ball valve is arranged between the cutting unit and the pressure-maintaining unit.

7. The device according to claim 1, further comprising: a pressurizing system used for maintaining and stabilizing an internal pressure of the device.

8. The device according to claim 1, wherein the data acquisition system comprises a pressure acquisition system and a temperature acquisition system, the pressure acquisition system comprises a plurality of pressure sensors and a pressure acquisition module, and the temperature acquisition system comprises a plurality of temperature sensors and a temperature acquisition module; and
the plurality of pressure sensors and the plurality of temperature sensors acquire real-time pressure values and temperature values of a plurality of positions in the device.

9. The device according to claim 8, wherein the data acquisition system further comprises a computer, and data processing and analyzing software installed in the computer.
